(19) United States
(12) Patent Application Publication
(10) Pub. No.: US 2025/0248638 A1
(43) Pub. Date: Aug. 7, 2025

(54) METHOD AND SYSTEM FOR INTEGRATING MYOGRAPHICAL SIGNALS AND MICROCONTROLLER

(71) Applicant: Purdue Research Foundation, West Lafayette, IN (US)

(72) Inventors: Ahmed Khaled Soliman, West Lafayette, IN (US); Gabriel Andres Torres Rivera, Lafayette, IN (US); Nina Mahmoudian, West Lafayette, IN (US); Mo Rastgaar, West Lafayette, IN (US)

(73) Assignee: Purdue Research Foundation, West Lafayette, IN (US)

(21) Appl. No.: 19/041,122

(22) Filed: Jan. 30, 2025

Related U.S. Application Data
(60) Provisional application No. 63/548,825, filed on Feb. 1, 2024.

(51) Int. Cl. A61B 5/397; A61B 5/30

(52) U.S. Cl. CPC A61B 5/397; A61B 5/30

(57) ABSTRACT

A processing system for electromyography signals includes one or more electrode input sets, one or more differential circuits each coupled to an associated one of the one or more electrode input sets and configured receive a corresponding positive input and a negative input of the corresponding electrode input set to thereby generate one or more differential signals each associated, one or more gain circuits each coupled to an associated one or more differential signals and configured to apply a selective gain to the associated one or more differential signals to thereby generate one or more gained signals, and one or more output pins each coupled to an associated one or more gained signals, to thereby generate a first one or more output myography signals, wherein the one or more output pins each configured to be coupled to a corresponding pin on a Micro:bit Edge Connector.

### CROSS-REFERENCE TO RELATED APPLICATIONS

The present non-provisional patent application is related to and claims the priority benefit of U.S. Provisional Patent Application Ser. 63/548,825, filed Feb. 1, 2024, the contents of which are hereby incorporated by reference in its entirety into the present disclosure.

### STATEMENT REGARDING GOVERNMENT FUNDING

This invention was made with government support under U.S. Pat. No. 2,133,028 awarded by the National Science Foundation. The government has certain rights in the invention.

### TECHNICAL FIELD

The present disclosure relates to an educational device to study biological signals, in particular to receiving and processing myography signals and processing those signals to a Micro:bit embedded system.

### BACKGROUND

This section introduces aspects that may help facilitate a better understanding of the disclosure. Accordingly, these statements are to be read in this light and are not to be understood as admissions about what is or is not prior art.

Robotic arms are common, but few take into account human bio-signals such as muscle activation via skin-mounted sensors. Specifically, myographical sensors are typically used to pick up muscle activation signals from a subject. These signals can be used to control prosthetic robots or robots detached from the subject for teaching purposes. For example, it is useful to have a teaching system for students to control a stand-alone robotic system that is controlled by a control system which receives its input from one or more myographical sensors. However, myographical sensors generally generate minute signal strength thus requiring a significant amount pre-processing conditioning.

There is an unmet need for a novel and convenient educational processing board that can receive myographical sensor signals and pre-process those signals to be used for downstream robotic control through an educational micro-controller unit, such as the Micro:bit system.

### SUMMARY

A processing system for electromyography signals that is coupled to a microprocessor is disclosed. The processing system includes one or more electrode input sets, each input set including a positive input, a negative input, and a ground input, each input configured to be coupled to a corresponding electrode positioned on a subject's muscle, one or more differential circuits each coupled to an associated one of the one or more electrode input sets and configured to generate a difference between the corresponding positive input and the negative input of the corresponding electrode input set to thereby generate one or more differential signals each associated with the corresponding one or more electrode input sets, one or more gain circuits each coupled to an associated one or more differential signals and configured to apply a selective gain to the associated one or more differential signals to thereby generate one or more gained signals, and one or more output pins each coupled to an associated one or more gained signals, to thereby generate a first one or more output myography signals, wherein the one or more output pins each configured to be coupled to a corresponding pin on a Micro:bit Edge Connector.

A myographical training system is also disclosed. The training system includes one or more electrode sets each configured to be placed on a subject and thus generate myographical signals via a positive electrode, a negative electrode, and a ground electrode, and a processing system. The processing system includes one or more electrode input sets, each input set including a positive input, a negative input, and a ground input, each input configured to be coupled to a corresponding electrode of a corresponding electrode set, one or more differential circuits each coupled to an associated one of the one or more electrode input sets and configured to generate a difference between the corresponding positive input and the negative input of the corresponding electrode input set to thereby generate one or more differential signals each associated with the corresponding one or more electrode input sets, one or more gain circuits each coupled to an associated one or more differential signals and configured to apply a selective gain to the associated one or more differential signals to thereby generate one or more gained signals, and one or more output pins each coupled to an associated one or more gained signals, to thereby generate a first one or more output myography signals.

### BRIEF DESCRIPTION OF DRAWINGS

FIGS. 1a and 1b provide a block diagram and a corresponding circuit diagram of a processing board, according to the present disclosure are provided, respectively.

FIG. 1c provides additional connectivity detail for a Micro:bit Edge Connector shown in FIG. 1a.

FIG. 2 is a schematic of a layout of the processing board of FIG. 1b.

FIGS. 3a, 3b, 3c, 3d, 3e, and 3f are graphs of voltage vs. time for various probe points in accordance with the input/output block and the circuit block, as discussed above with reference to FIGS. 1a and 1b.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 15%, within 10%, within 5%, or within 1% of a stated value or of a stated limit of a range.

In the present disclosure, the term “substantially” can allow for a degree of variability in a value or range, for example, within 85%, within 90%, within 95%, or within 99% of a stated value or of a stated limit of a range.

A novel system and method are disclosed herein that can receive myographical sensor signals and pre-process those signals to be used for downstream robotic control. Towards this end, a processing board is disclosed herein that is configured to receive raw myographical signals from one or more myographical sensors as inputs and generate signals that can be used for controlling one or more motors disposed in one or more robots as outputs.

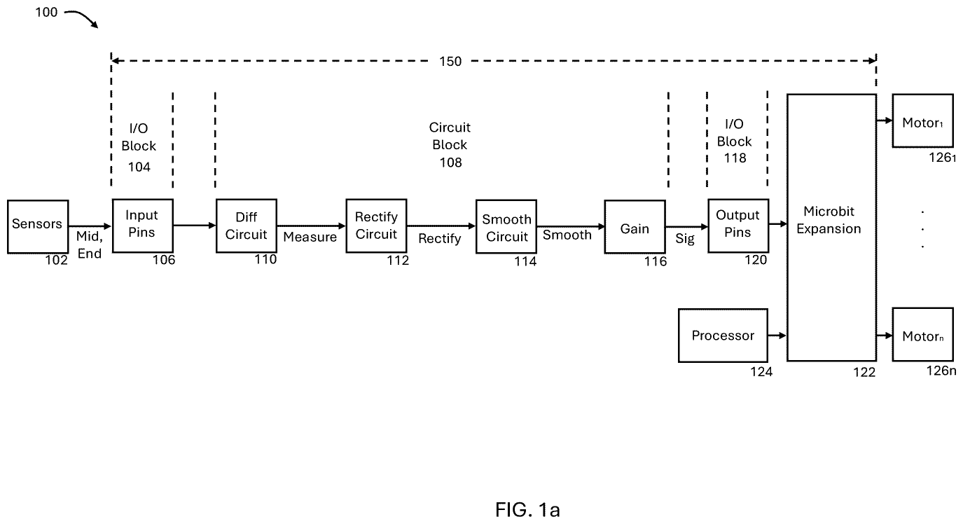

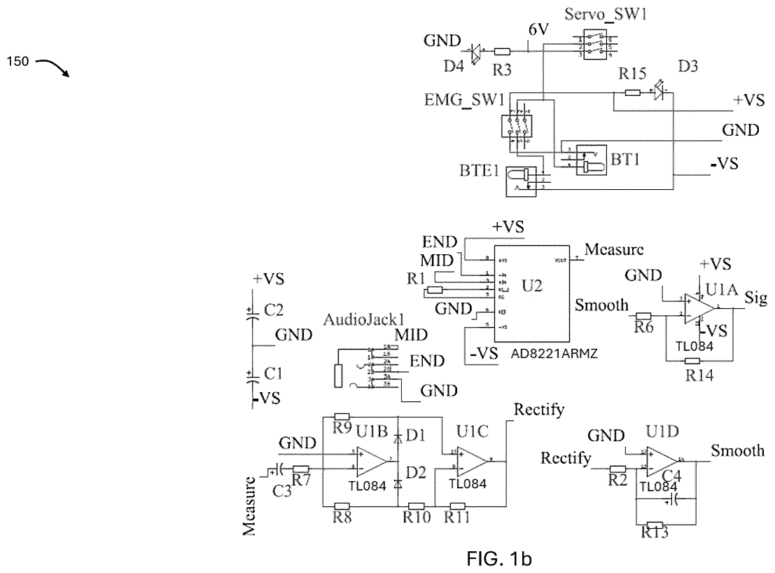

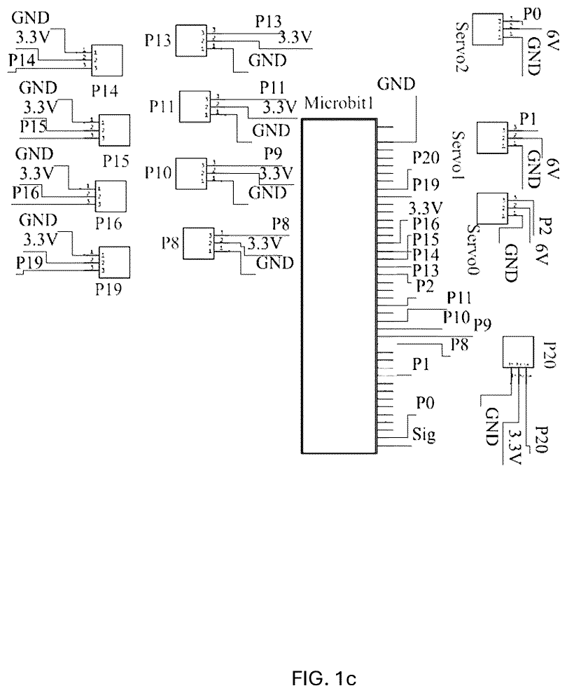

Referring to FIGS. 1a and 1b, a block diagram of a system 100 and a corresponding processing board 150 shown in FIG. 1b, according to the present disclosure, are provided, respectively. The processing board 150 includes major blocks including a first input/output (I/O) block 104 receiving inputs from sensors 102, a circuit block 108, and second I/O block 118. The second I/O block 118 includes electrode input sets and output pins 120. The circuit block 108 includes various circuits including differential circuits 110, rectifying circuits 112, smoothing circuits 114, and gain circuits 116. In particular, the processing board 150 includes one or more electrode input pin sets 106. Each of these input pin sets 106 includes a positive input (e.g., Mid), a negative input (e.g., End), and a ground input. Each input is configured to be coupled to a corresponding electrode positioned on a subject's muscle. For example, a first electrode that is coupled to the positive input may be coupled to a subject's skin about an upper portion of the subject's bicep muscle on a first arm; a second electrode that is coupled to the negative input may be coupled to a subject's skin about a lower portion of the subject's bicep of the first arm, and a third electrode that is coupled to the ground input may be coupled to a subject's skin away from the subject's bicep of the first arm, e.g., on the second arm of the subject.

The processing board 150 also includes one or more differential circuits 110 each coupled to an associated one of the one or more electrode input sets. Each of these one or more differential circuits 110 is configured to generate a difference signal between the corresponding positive input and the negative input of the corresponding electrode input set based on the ground signal generated from the third electrode to thereby generate one or more differential signals each associated with the corresponding one or more electrode input sets. An example of the one or more differential circuits is the ANALOG DEVICES INC.® AD8221ARMZ which is an instrumentation amplifier. The output of the differential circuits is specified as “Measure.”

The processing board 150 also includes one or more rectifying circuits 112 each configured to be coupled to a corresponding of the one or more differential circuits 110 and their associated one or more differential signals to thereby generate one or more rectified signals. An example of the rectifier circuit is a back-to-back operational amplifier (e.g., made by TEXAS INSTRUMENTS®, e.g., 2×TL084) which is a JFET-input operational amplifier. The output of the rectifying circuits is specified as “Rectify.”

The processing board also includes one or more smoothing circuits 114, each coupled to a corresponding of the one or more rectified signals from a corresponding rectify circuit 112 to thereby generate a one or more smoothed signals. An example of the smoothing circuit is an operational amplifier (e.g., made by TEXAS INSTRUMENTS®, e.g., TL084) which is a JFET-input operational amplifier. The output of the smoothing circuits is specified as “Smooth.”

The processing board 150 also includes one or more gain circuits 116 each coupled to a corresponding of the one or more smoothed signals from a corresponding smoothing circuit 114 and configured to apply a selective gain thereto to thereby generate one or more gained signals. An example of the gain circuit is an operational amplifier (e.g., made by TEXAS INSTRUMENTS®, e.g., TL084). The output of the gain circuits is specified as “Sig.”

The processing board 150 also includes one or more output pins 120 each coupled to a corresponding of the one or more gained signals from a corresponding gain circuit 116, to thereby generate one or more output myography signals. The one or more output pins are coupled to a Micro:bit Edge Connector 122. The Micro:bit Edge Connector is also coupled to a processor 124, e.g., a microprocessor with onboard or offboard non-transient memory carrying instructions which when executed by the processor receives the output of the one or more gain circuits 116, i.e., identified as “Sig,” and carries out said instructions. The processor then outputs one or more motor outputs (identified as Motor₁ 126₁ . . . . Motorₙ 126ₙ) which are outputs to robots, e.g., teaching robots or prosthetics that are actuated by the one or more motor outputs based on the measured “Sig”.

It should be appreciated that each of the aforementioned circuit blocks (i.e., the differential circuits 110, the rectifying circuits 112, the smoothing circuits 114, and the gain circuits 116) can be provided alone or in combination with the other circuits mentioned herein. For example, the differential circuits 110 may be combined with the gain circuits 116 without inclusion of the rectifying circuits 112 and without providing the smoothing circuits 114 to provide one or more differential-gain signals. Alternatively, the differential circuits 110 may be combined with the rectifying circuits 112 and the gain circuits 116 but without the smoothing circuits 114 to generate differential-rectified-gained signals. Other combinations of circuits are also possible. Still alternatively yet, the differential circuits 110 may be avoided altogether wherein each of the electrode signals is further processed by any combination of the rectifying circuits 112, the smoothing circuits 114, and the gain circuits 116 to thereby generate modified output signals. Referring to FIG. 1b, the above-described signals (i.e., Measure, Rectify, Smooth, and Sig) are shown as outputs of individual sub-circuit in accordance with the above description.

Referring to FIG. 1c, additional connectivity detail is shown for the Micro:bit Edge Connector 122.

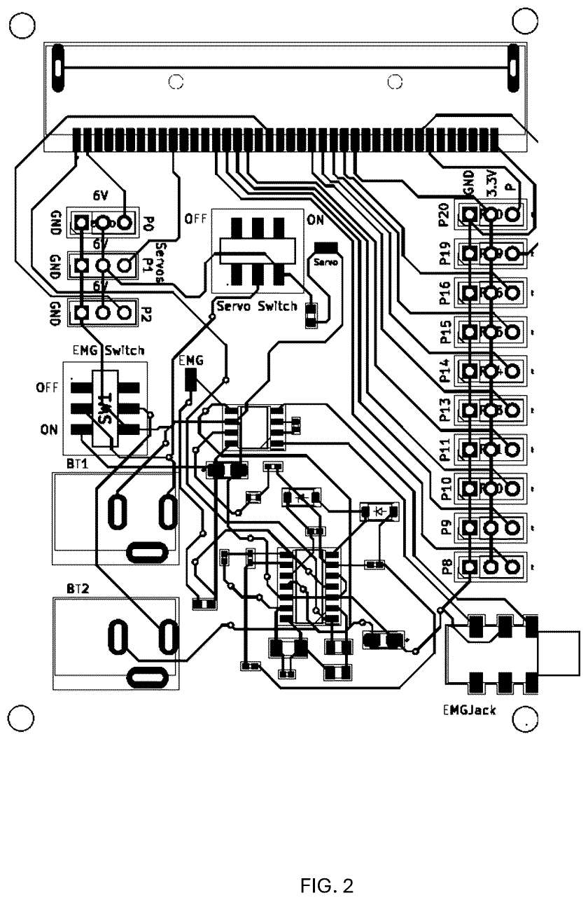

Referring to FIG. 2, a schematic of a layout of the processing board 150 is provided.

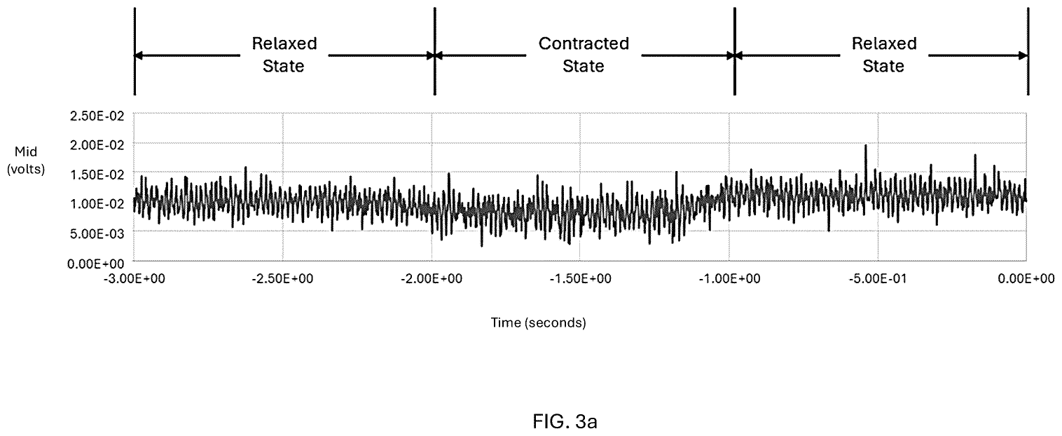

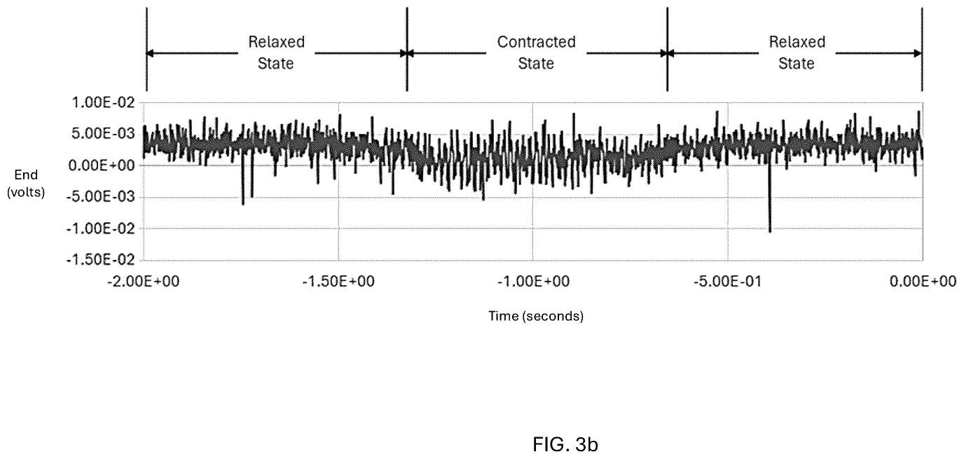

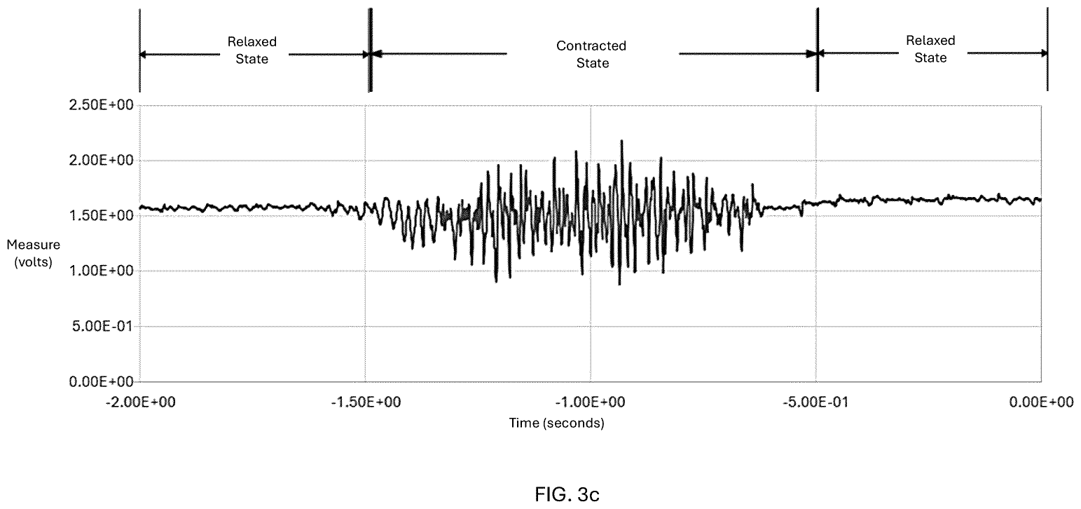

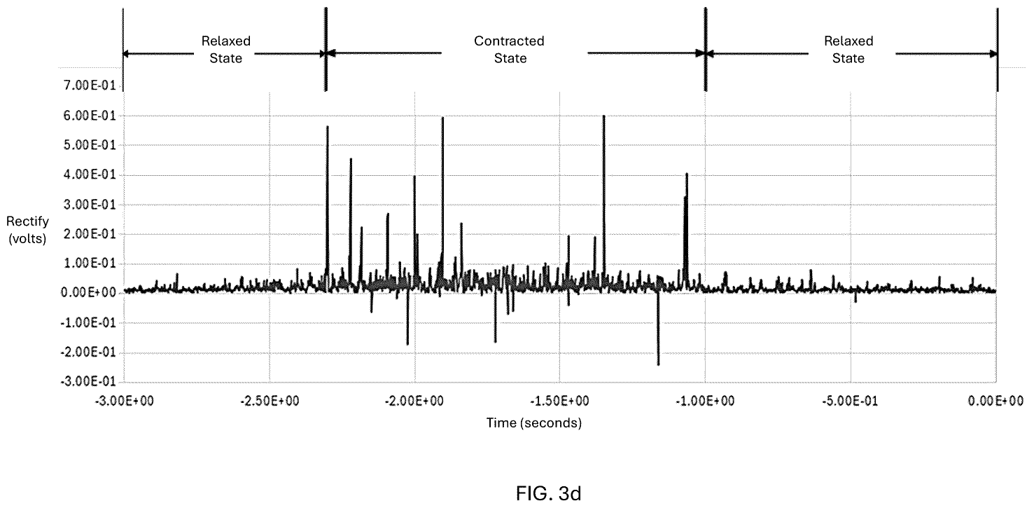

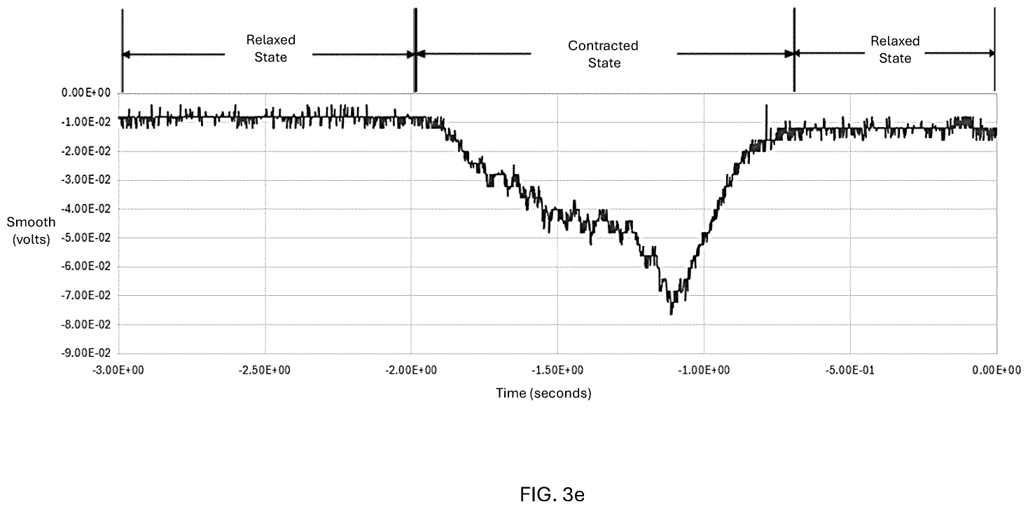

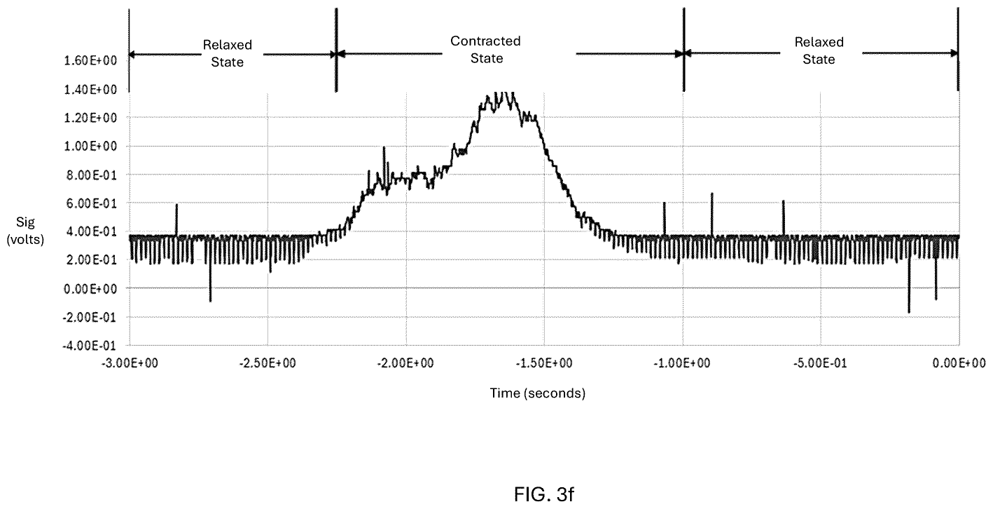

Referring to FIGS. 3a-3f, graphs of voltage vs. time is provided for various probe points in accordance with the input/output block and the circuit block, as discussed above with reference to FIGS. 1a and 1b. For example FIGS. 3a and 3b are graphs of voltage vs. time for Mid and end signals (i.e., raw myographical signals from the electrodes as discussed above) during relaxed states of the muscle and also during a contracted state of the muscle. As discussed above, the Mid and End signals are subtracted and gained via the one or more differential circuits thus generating the Measure signal which is shown in FIG. 3c, which is a graph of one Measure signal in voltage vs. time in seconds, shown again in the relaxed states and the contracted state. The output of the one or more rectifying circuits is shown in FIG. 3d, which is a graph of one Rectify signal in voltage vs. time in seconds, shown again in the relaxed states and the contracted state. The output of the one or more smoothing circuits is shown in FIG. 3e, which is a graph of one Smooth signal in voltage vs. time in seconds, shown again in the relaxed states and the contracted state. The output of the one or more gain circuits is shown in FIG. 3f, which is a graph of one Sig signal in voltage vs. time in seconds, shown again in the relaxed states and the contracted state.

As discussed above, the processor 124 that is coupled to the Micro:bit Edge Connector 122 is configured to receive the output of the one or more gain circuits 116 (i.e., Sig signals) via the Micro:bit Edge Connector 122 and execute software held in a non-transitory memory. Specifically, the processor 124 is configured to: 1) establish a relaxed state from each of the one or more gain circuit outputs (i.e., Sig signals), associated with the above-described relaxed states of the subject's muscle; 2) establish a contracted state from each of the one or more gain circuit outputs (i.e., Sig signals), associated with the above-described contracted state of the subject's muscle; 3) apply a first moving window to each of the one or more gain circuits outputs (i.e., Sig signals) associated with both the established relaxed state and the established contracted state to thereby establish a working window associated with each state; 4) determine a first maximum value associated with a maximum value of the relaxed state moving window and a second maximum value associated with a maximum value of the contracted state moving window; 5) determine a difference between the first maximum value and the second maximum value thereby generating a third maximum value; 6) linearly transform the third maximum value to a predetermined range for an associated motor, e.g., to thereby generate linearly scaled values; and outputting values associated with the linearly transformation via the Micro:bit Edge Connector 122 as Motor₁ 126₁ . . . . Motorₙ 126ₙ outputs.

Those having ordinary skill in the art will recognize that numerous modifications can be made to the specific implementations described above. The implementations should not be limited to the particular limitations described. Other implementations may be possible.

1. A processing system for electromyography signals that is coupled to a microprocessor, comprising:
one or more electrode input sets, each input set including a positive input, a negative input, and a ground input, each input configured to be coupled to a corresponding electrode positioned on a subject's muscle;
one or more differential circuits each coupled to an associated one of the one or more electrode input sets and configured to generate a difference between the corresponding positive input and the negative input of the corresponding electrode input set to thereby generate one or more differential signals each associated with the corresponding one or more electrode input sets;
one or more gain circuits each coupled to an associated one or more differential signals and configured to apply a selective gain to the associated one or more differential signals to thereby generate one or more gained signals; and
one or more output pins each coupled to an associated one or more gained signals, to thereby generate a first one or more output myography signals, wherein the one or more output pins each configured to be coupled to a corresponding pin on a Micro:bit Edge Connector, the one or more output myography signals is then read by a microprocessor coupled to the Micro:bit Edge Connector.

2. The processing system of claim 1, wherein a first one or more of the one or more differential signals each is rectified by a first associated rectifier circuit to thereby generate a first one or more rectified signals prior to being coupled to a first one or more associated gain circuits, the output of the first one or more gain circuits thereby generating a first one or more rectified-gained signals, wherein a first one or more associated output pins each is coupled to the first one or more rectified-gained signals to thereby generated a second one or more output myography signals.

3. The processing system of claim 2, wherein a second one or more of the one or more differential signals each is smoothed by a first associated smoothing circuit to thereby generate a first one or more smoothed signals prior to being coupled to a second one or more associated gain circuits, the output of the second one or more gain circuits thereby generating one or more smoothed-gained signals, wherein a second one or more associated output pins each is coupled to the one or more smoothed-gained signals to thereby generate a third one or more output myography signals.

4. The processing system of claim 2, wherein a third one or more of the one or more differential signals each is rectified by a second associated rectifying circuit to thereby generate a second one or more rectified signals, the second one or more rectified signals each is smoothed by an associated smoothing circuit prior to being coupled to a second one or more associated gain circuits, the output of the second one or more gain circuits thereby generating one or more rectified-smoothed-gained signals, wherein a second one or more associated output pins each is coupled to the one or more rectified-smoothed-gained signals to thereby generated a third one or more output myography signals.

5. The processing system of claim 4, wherein one or more of the one or more rectified-smoothed-gained signals is processed as an associated motor drive signals to thereby generate one or more motor drive signal, each of the one or more motor drive signals provided as an associated one or more motor drive outputs.

6. The processing system of claim 5, wherein a Micro:bit processor executing software on a non-transient memory is configured to:
establish a relaxed state whereby each of the one or more rectified-smoothed-gained signals is associated with a relaxed state of the subject's muscle;
establish a contracted state whereby each of the one or more rectified-smoothed-gained signals is associated with a contracted state of the subject's muscle;
apply a first moving window to each of the one or more rectified-smoothed-gained signals associated with both relaxed state and contracted state to thereby establish a working window associated with each state;
determine a first maximum value associated with a maximum value of the relaxed state moving window and a second maximum value associated with a maximum value of the contracted state moving window;
determine a difference between the first maximum value and the second maximum value thereby generating a third maximum value;
linearly transform the third maximum value to a predetermined range for an associated motor to thereby generate linearly scaled values; and
outputting values associated with the linear transformation.

7. The processing system of claim 1, wherein each of the one or more differential circuits comprises at least one differential operational amplifier.

8. The processing system of claim 1, wherein each of the one or more gain circuits comprises at least one gain operational amplified.

9. The processing system of claim 4, wherein each of the second associated rectifying circuits comprises at least two back-to-back operational amplifiers.

10. The processing system of claim 4, wherein each of the associated smoothing circuits comprises at least one smoothing operational amplifier.

11. The processing system of claim 1, further comprising a power source for providing electrical power.

12. The processing system of claim 11, wherein the power source is a battery.

13. A myographical training system, comprising:
one or more electrode sets each configured to be placed on a subject and thus generate myographical signals via a positive electrode, a negative electrode, and a ground electrode;
a processing system comprising:
one or more electrode input sets, each input set including a positive input, a negative input, and a ground input, each input configured to be coupled to a corresponding electrode of a corresponding electrode set;
one or more differential circuits each coupled to an associated one of the one or more electrode input sets and configured to generate a difference between the corresponding positive input and the negative input of the corresponding electrode input set to thereby generate one or more differential signals each associated with the corresponding one or more electrode input sets;
one or more gain circuits each coupled to an associated one or more differential signals and configured to apply a selective gain to the associated one or more differential signals to thereby generate one or more gained signals; and
one or more output pins each coupled to an associated one or more gained signals, to thereby generate a first one or more output myography signals.

14. The myographical training system of claim 13, wherein a first one or more of the one or more differential signals each is rectified by a first associated rectifier circuit to thereby generate a first one or more rectified signals prior to being coupled to a first one or more associated gain circuits, the output of the first one or more gain circuits thereby generating a first one or more rectified-gained signals, wherein a first one or more associated output pins each is coupled to the first one or more rectified-gained signals to thereby generated a second one or more output myography signals.

15. The myographical training system of claim 14, wherein a second one or more of the one or more differential signals each is smoothed by a first associated smoothing circuit to thereby generate a first one or more smoothed signals prior to being coupled to a second one or more associated gain circuits, the output of the second one or more gain circuits thereby generating one or more smoothed-gained signals, wherein a second one or more associated output pins each is coupled to the one or more smoothed-gained signals to thereby generate a third one or more output myography signals.

16. The myographical training system of claim 14, wherein a third one or more of the one or more differential signals each is rectified by a second associated rectifying circuit to thereby generate a second one or more rectified signals, the second one or more rectified signals each is smoothed by an associated smoothing circuit prior to being coupled to a second one or more associated gain circuits, the output of the second one or more gain circuits thereby generating one or more rectified-smoothed-gained signals, wherein a second one or more associated output pins each is coupled to the one or more rectified-smoothed-gained signals to thereby generated a third one or more output myography signals.

17. The myographical training system of claim 16, further comprising one or more motors, wherein one or more of the one or more rectified-smoothed-gained signals is processed as an associated motor drive signals to thereby generate one or more motor drive signal for the associated motor.

18. The myographical training system of claim 17, wherein a processor executing software on a non-transient memory is configured to:
establish a relaxed state whereby each of the one or more rectified-smoothed-gained signals is associated with a relaxed state of the subject's muscle;
establish a contracted state whereby each of the one or more rectified-smoothed-gained signals is associated with a contracted state of the subject's muscle;
apply a first moving window to each of the one or more rectified-smoothed-gained signals associated with both relaxed state and contracted state to thereby establish a working window associated with each state;
determine a first maximum value associated with a maximum value of the relaxed state moving window and a second maximum value associated with a maximum value of the contracted state moving window;
determine a difference between the first maximum value and the second maximum value thereby generating a third maximum value;
linearly transform the third maximum value to a predetermined range for an associated motor to thereby generate linearly scaled values; and
outputting values associated with the linear transformation.

19. The myographical training system of claim 13, wherein each of the one or more differential circuits comprises at least one differential operational amplifier.

20. The myographical training system of claim 13, wherein each of the one or more gain circuits comprises at least one gain operational amplified.

21. The myographical training system of claim 16, wherein each of the second associated rectifying circuits comprises at least two back-to-back operational amplifiers.

22. The myographical training system of claim 16, wherein each of the associated smoothing circuits comprises at least one smoothing operational amplifier.
